(19) United States
(12) Patent Application Publication
(10) Pub. No.: US 2007/0149593 A1
(43) Pub. Date: Jun. 28, 2007

(54) PHARMACEUTICAL FORMULATION FOR DELIVERY OF RECEPTOR TYROSINE KINASE INHIBITING (RTKi) COMPOUNDS TO THE EYE

(75) Inventors: Malay Ghosh, Fort Worth, TX (US); Wesley Wehsin Han, Arlington, TX (US); Way-Yu Lin, Arlington, TX (US); David P. Bingaman, Weatherford, TX (US); Stella M. Robertson, Fort Worth, TX (US)

Correspondence Address: ALCON, IP LEGAL, TB4-8, 6201 SOUTH FREEWAY, FORT WORTH, TX, 76134, US

(73) Assignee: Alcon, Inc., Hunenberg (CH)

(21) Appl. No.: 11/612,744

(22) Filed: Dec. 19, 2006

Related U.S. Application Data
(60) Provisional application No. 60/753,713, filed on Dec. 23, 2005.

(51) Int. Cl. A61K 31/416; A61K 31/08

(52) U.S. Cl. 514/406; 514/723

(57) ABSTRACT

The present invention relates to development of efficacious intravitreal pharmaceutical compositions comprising a poorly water soluble agent with anti-angiogenic and/or anti vascular leakage properties in a therapeutically effective amount and a co-solvent in a suitable amount to treat or prevent diseases due to ocular neovascularization and enhanced vascular permeability. Other aspects of the invention details the development of efficacious compositions for the treatment of the said diseases via periocular, topical and oral administration.

This application claims priority to U.S. provisional application Ser. No. 60/753,713 filed Dec. 23, 2005.

### BACKGROUND OF THE INVENTION

1. Field of the Invention

The present invention relates to unique compositions containing compounds with poor solubility and methods useful for treating pathological states that arise or are exacerbated by ocular inflammation, angiogenesis and vascular leakage such as AMD, DR, diabetic macular edema etc., and more specifically, to compositions containing at least one anti-angiogenic, anti-inflammatory or anti-vascular leakage agent for use in treating ocular disorders.

2. Description of the Related Art

Abnormal neovascularization or angiogenesis and enhanced vascular permeability are major causes for many ocular disorders including age-related macular degeneration (AMD), retinopathy of prematurity (ROP), ischemic retinal vein occlusions and diabetic retinopathy (DR). AMD and DR are among the most common cause of severe, irreversible vision loss. In these and related diseases, such as retinal vein occlusion, central vision loss is secondary to angiogenesis, the development of new blood vessels from pre-existing vasculature, and alterations in vascular permeability properties.

The angiogenic process is known by the activation of quiescent endothelial cells in pre-existing blood vessels. The normal retinal circulation is resistant to neovascular stimuli, and very little endothelial cell proliferation takes place in the retinal vessels. While there appear to be many stimuli for retinal neovascularization, including tissue hypoxia, inflammatory cell infiltration and penetration barrier breakdown, all increase the local concentration of cytokines (VEGF, PDGF, FGF, TNF, IGF etc.), integrins and proteinases resulting in the formation of new vessels, which then disrupt the organizational structure of the neural retina or break through the inner limiting membranes into the vitreous. Elevated cytokine levels can also disrupt endothelial cell tight junctions, leading to an increase in vascular leakage and retinal edema, and disruption of the organizational structure of the neural retina. Although VEGF is considered to be a major mediator of inflammatory cell infiltration, endothelial cell proliferation and vascular leakage, other growth factors, such as PDGF, FGF, TNF, and IGF etc., are involved in these processes. Therefore, growth factor inhibitors can play a significant role in inhibiting retinal damage and the associated loss of vision upon local delivery in the eye or via oral dosing.

There is no cure for the diseases caused by ocular neovascularization and enhanced vascular permeability. The current treatment procedures of AMD include laser photocoagulation and photodynamic theraphy (PDT). The effects of photocoagulation on ocular neovascularization and increased vascular permeability are achieved only through the thermal destruction of retinal cells. PDT usually requires a slow infusion of the dye, followed by application of non-thermal laser-light. Treatment usually causes the abnormal vessels to temporarily stop or decrease their leaking. PDT treatment may have to be repeated every three months up to 3 to 4 times during the first year. Potential problems associated with PDT treatment include headaches, blurring, and decreased sharpness and gaps in vision and, in 1-4% of patients, a substantial decrease in vision with partial recovery in many patients. Moreover, immediately following PDT treatment, patients must avoid direct sunlight for five (5) days to avoid sunburn. Recently, a recombinant humanized IgG monoclonal antibody fragment was approved (ranibizumab) in the US for treatment of patients with age-related macular degeneration. This drug is typically administered via intravitreal injection once a month.

Many compounds that may be considered potentially useful in treating ocular neovascularization and enhanced vascular permeability-related and other disorders, are poorly soluble in water. A poorly water soluble compound is a substance that is not soluble at a therapeutically effective concentration in an aqueous physiologically acceptable vehicle. Aqueous solubility is an important parameter in formulation development of a poorly water soluble compound. What is needed is a formulation that provides increased solubility of the compound while also providing sufficient bioavailability of the compound so as to maintain its therapeutic potential.

The present invention provides safe and effective formulations for ocular administration of poorly soluble compounds for the treatment of ocular diseases caused by endothelial cell proliferation, vascular leakage, inflammation and angiogenesis.

### SUMMARY OF THE INVENTION

The present invention overcomes these and other drawbacks of the prior art by providing compositions for treating ocular diseases due to angiogenesis and increased vascular permeability. Within one aspect of the present invention, intravitreal compositions having pan-retinal distribution are provided. The compositions of the invention include (a) an agent capable of controlling neovascularization or ocular inflammation or vascular leakage, (b) a suitable co-solvent in appropriate amount, (c) a surfactant, (d) a suspending agent, and (e) buffer. A wide variety of molecules may be utilized within the scope of present invention, as well as various suitable suspending agents and co-solvents. The amount of co-solvent plays a very important role on the efficacy of the formulation upon local delivery.

In another embodiment, posterior juxtascleral (PJ) and periocular (PO) formulations (PO) containing (a) an agent capable of controlling neovascularization or ocular inflammation or vascular leakage, (b) a suitable amount of co-solvent, (c) tonicity agents so that tonicity is around 300 mOsm/kg, (d) a buffer, (e) a suspending agent, and (f) a surfactant are provided.

In a separate embodiment, efficacious formulations for oral dosing are prepared using (a) a suitable amount of an agent capable of controlling neovascularization or ocular inflammation or vascular leakage, (b) a suspending agent, (c) a surfactant, and (d) a co-solvent. This formulation is able to circumvent the blood-retinal barrier to provide a therapeutically effective concentration of the active drug to the posterior part of the eye.

In yet another embodiment, the present invention provides formulations for topical ocular dosing, which include (a) a therapeutically effective amount of an agent capable of controlling neovascularization or ocular inflammation or vascular leakage, (b) a suspending agent, (c) a surfactant, and (d) a co-solvent. This formulation is able to circumvent blood-retinal barrier to provide a therapeutically effective concentration of the active drug to the posterior part of the eye.

### BRIEF DESCRIPTION OF THE DRAWINGS

The following drawing forms part of the present specification and is included to further demonstrate certain aspects of the present invention. The invention may be better understood by reference to this drawing in combination with the detailed description of specific embodiments presented herein.

FIG. 1 shows inhibition of preretinal neovascularization in the rat OIR model upon single intravitreal injection of RTKi (N-[4-(3-amino-1H-indazol-4-yl)phenyl]-N′-(2-fluoro-5-methylphenyl)urea) formulations, prepared in different vehicles. The formulation containing 10% PEG 400 exhibits complete inhibition of preretinal neovascularization.

FIG. 2 shows the effect of PEG 400 concentration on the efficacy of 1% RTKi (N-[4-(3-amino-1H-indazol-4-yl)phenyl]-N′-(2-fluoro-5-methylphenyl)urea) intravitreal formulation in the rat OIR model. The formulation with 10% PEG 400 shows 100% inhibition whereas formulations with 5% and 0% PEG 400 show 67% and 34% inhibition, respectively.

FIG. 3 shows dissected rat retina treated with 1% RTKi (N-[4-(3-amino-1H-indazol-4-yl)phenyl]-N′-(2-fluoro-5-methylphenyl)urea) intravitreal formulation without PEG 400. Significant neovascularization is observed.

FIG. 4 shows dissected rat retina treated with 1% RTKi (N-[4-(3-amino-1H-indazol-4-yl)phenyl]-N′-(2-fluoro-5-methylphenyl)urea) intravitreal formulation containing 10% PEG 400. Complete inhibition of preretinal neovascularization is observed.

FIG. 5 shows results of a dose response study of RTKi (N-[4-(3-amino-1H-indazol-4-yl)phenyl]-N′-(2-fluoro-5-methylphenyl)urea) intravitreal formulations in the rat OIR model.

FIG. 6. shows the efficacy of RTKi (N-[4-(3-amino-1H-indazol-4-yl)phenyl]-N′-(2-fluoro-5-methylphenyl)urea) formulation on VEGF induced retinal vascular leakage following a single intravitreal injection in rabbits.

FIG. 7. shows inhibition of preretinal neovascularization by RTKi (N-[4-(3-amino-1H-indazol-4-yl)phenyl]-N′-(2-fluoro-5-methylphenyl)urea) formulations, prepared in PEG 400/Polysorbate 80/HPMC vehicles in the rat OIR model upon single intravitreal injection. The formulation resulted complete inhibition of preretinal neovascularization.

FIG. 8. shows a dose response study in the rat OIR model using modified RTKi (N-[4-(3-amino-1H-indazol-4-yl)phenyl]-N′-(2-fluoro-5-methylphenyl)urea) intravitreal formulations with enhanced physical stability.

FIG. 9. shows inhibition of laser induced CNV in the domestic pig model upon intravitreal injection of RTKi (N-[4-(3-amino-1H-indazol-4-yl)phenyl]-N′-(2-fluoro-5-methylphenyl)urea) formulations.

FIG. 10. shows a dose response study of RTKi (N-[4-(3-amino-1H-indazol-4-yl)phenyl]-N′-(2-fluoro-5-methylphenyl)urea) formulations on the laser induced CNV mouse model.

FIG. 11. shows the effect of periocular (PO) administration of 5% RTKi (N-[4-(3-amino-1H-indazol-4-yl)phenyl]-N′-(2-fluoro-5-methylphenyl)urea) formulation in the domestic pig model.

FIG. 12. shows the effect of RTKi (N-[4-(3-amino-1H-indazol-4-yl)phenyl]-N′-(2-fluoro-5-methylphenyl)urea) formulation (20 mg/kg/day) given via oral gavage on VEGF induced retinal vascular permeability in the adult rat.

FIG. 13. shows the efficacy of RTKi (N-[4-(3-amino-1H-indazol-4-yl)phenyl]-N′-(2-fluoro-5-methylphenyl)urea) formulation via oral gavage on laser induced CNV model in adult mouse.

FIG. 14. shows regression of existing choroidal neovascularization (CNV) with oral gavage of RTKi (N-[4-(3-amino-1H-indazol-4-yl)phenyl]-N′-(2-fluoro-5-methylphenyl)urea) formulation in the laser induced mouse CNV model.

### DETAILED DESCRIPTION OF PREFERRED EMBODIMENTS

As noted above, the present invention provides compositions that contain an antiangiogenic or anti-inflammatory or anti vascular-leakage agent for use in the treatment of ocular angiogenesis and vascular leakage related disorders. The compositions of the invention are useful in preventing or inhibiting neovascularization and vascular leakage associated with such ocular disorders. In some cases, the compositions of the invention cause regression of neovascularization.

Briefly, within the context of the present invention, active agents should be understood to be any molecule, either synthetic or naturally occurring, which acts to inhibit vascular growth, reduce vascular permeability, and/or decrease inflammation. In particular, the present invention provides compositions comprising an insoluble or poorly soluble, active agent in a therapeutically effective amount for delivery of active agent to the eye of a patient suffering from ocular neovascularization or ocular vascular leakage-related disorders.

It is contemplated that any active agent that is poorly water soluble may be included in the compositions of the present invention. For example, anti-angiogenic agents, anti-inflammatory agents, or anti-vascular permeability agents are useful in the compositions of the invention.

Preferred anti-angiogenic agents include, but are not limited to, receptor tyrosine kinase inhibitors (RTKi), in particular, those having a multi-targeted receptor profile such as that described in further detail herein; angiostatic cortisenes; MMP inhibitors; integrin inhibitors; PDGF antagonists; antiproliferatives; HIF-1 inhibitors; fibroblast growth factor inhibitors; epidermal growth factor inhibitors; TIMP inhibitors; insulin-like growth factor inhibitors; TNF inhibitors; antisense oligonucleotides; anti-VEGF antibody, VEGF trap, NSAID, steroids, SiRNA etc., and prodrugs of any of the aforementioned agents. The preferred anti-angiogenic agent for use in the present invention is a multi-targeted receptor tyrosine kinase inhibitor (RTKi). Most preferred are RTKi's with multi-target binding profiles, such as AL-39324, N-[4-(3-amino-1H-indazol-4-yl)phenyl]-N′-(2-fluoro-5-methylphenyl)urea, having the binding profile substantially similar to that listed in Table 1. Additional multi-targeted receptor tyrosine kinase inhibitors contemplated for use in the compositions of the present invention are described in U.S. Application Serial No. 2004/0235892, incorporated herein by reference. As used herein, the term “multi-targeted receptor tyrosine kinase inhibitor” refers to a compound having a receptor binding profile exhibiting selectivity for multiple receptors shown to be important in angiogenesis, such as the profile shown in Table 1, and described in co-pending U.S. application No. 2006/0189608, incorporated herein by reference. More specifically, the preferred binding profile for the multi-targeted receptor tyrosine kinase inhibitor compounds for use in the compositions of the present invention is KDR (VEGFR2), Tie-2 and PDGFR.

TABLE 1
Kinase Selectivity Profile of a RTK Inhibitor

| KDR | FLT1 | FLT4 | PDGFR | CSF1R | KIT | FLT3 | TIE2 | FGFR | EGFR | SRC |
|---|---|---|---|---|---|---|---|---|---|---|
| 4 | 3 | 190 | 66 | 3 | 14 | 4 | 170 | >12,500 | >50,000 | >50,000 |

All data reported as IC₅₀ values for kinase Inhibition in cell-free enzymatic assays; ND denotes no data. Values determined @ 1 mM ATP.

Other agents which will be useful in the compositions and methods of the invention include anti-VEGF antibody (i.e., bevacizumab or ranibizumab); VEGF trap; siRNA molecules, or a mixture thereof, targeting at least two of the tyrosine kinase receptors having IC₅₀ values of less than 200 nM in Table 1; glucocorticoids (i.e., dexamethasone, fluoromethalone, medrysone, betamethasone, triamcinolone, triamcinolone acetonide, prednisone, prednisolone, hydrocortisone, rimexolone, and pharmaceutically acceptable salts thereof, prednicarbate, deflazacort, halomethasone, tixocortol, prednylidene (21-diethylaminoacetate), prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, and deacylcortivazol oxetanone); Naphthohydroquinone antibiotics (i.e., Rifamycin); and NSAIDs (i.e., nepafenac, amfenac).

The formulations described herein may be delivered topically, orally, via intravitreal injection, or via posterior juxtascleral, periocular and topical ocular routes. Preferred co-solvents for use in the compositions of the present invention include ethylene glycol, propylene glycol, N-methylpyrrolidinone, 2-pyrrolidinone, 3-pyrrolidinol, 1,4-butanediol, dimethylglycol monomethylether, diethyleneglycol monomethyl ether, solketal, glycerol, polyethylene glycol, polypropylene glycol etc. Especially preferred is polyethylene glycol 200 to 2500 (PEG 200 to PEG 2500). The most preferred co-solvent for use in the formulations of the present invention is PEG 400 or PEG 2000. The co-solvent will typically be present in the intravitreal formulation of the invention in an amount from 1% to 30%. Preferably, the compositions of the invention will contain from 5% to 20% co-solvent. Most preferably, the composition for intravitreal injection will contain 10% co-solvent. The composition for posterior juxtascleral, periocular and topical administration will most preferably contain 5% co-solvent.

Polyethylene glycols (PEG) have a general chemical formula HOCH₂(CH₂OCH₂)ₙCH₂OH. They are nonvolatile, water soluble or water-miscible compounds and chemically inert, varying in molecular weights from several hundred to several thousand. They are liquids or waxy solids identified by numbers which are an approximate indication of molecular weight. PEG 400 is a liquid, while PEG 2500 is a waxy solid.

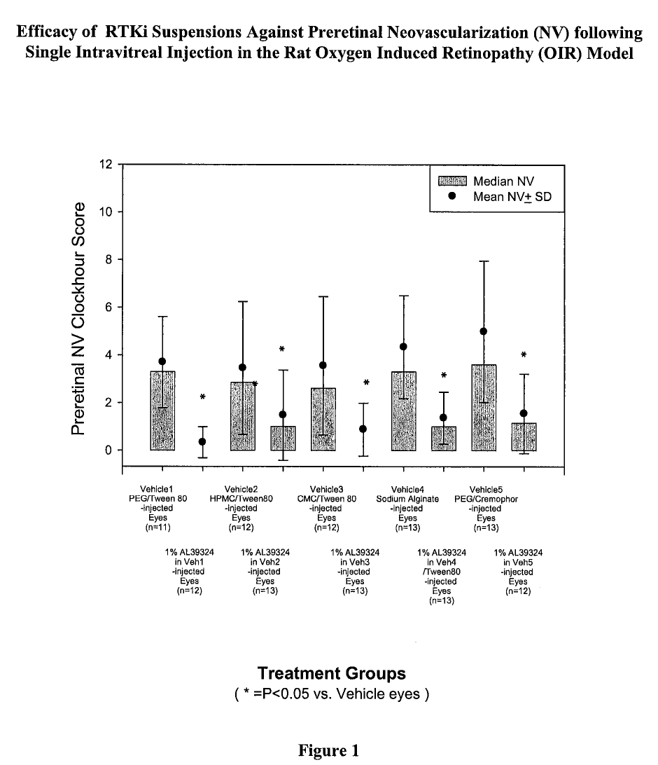

Polyethylene glycol, with a molecular weight from 200 to 2500 (PEG 200 to PEG 2500), is identified as a key excipient for efficacious intravitreal, periocular and posterior juxtascleral formulations. PEG 400 based formulations containing a RTKi having a multi-targeted receptor binding profile show excellent efficacy (100% inhibition) and almost no side effects upon single intravitreal injection compared to other formulations prepared from other polymers (FIG. 1) in the rat OIR model. Formulations containing PEG 2000 also exhibit excellent efficacy, as shown in the examples herein. Although both CMC and sodium alginate based formulations showed good inhibition, significant side effects including retinal hemorrhage and inflammation were observed with their application. HPMC based formulations (without PEG 400) did not cause side effects, but failed to show 100% inhibition. Formulation containing both HPMC and 10% PEG 400 showed excellent inhibition along with no side effect. The efficacy of formulations containing polysorbate 80 appears to be superior to the formulations containing Cremophore EL (FIG. 1).

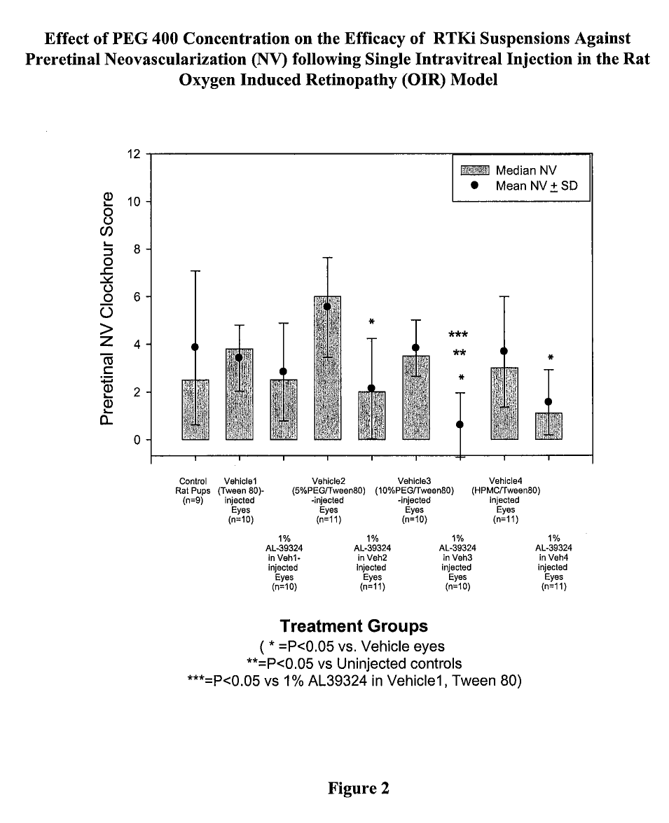

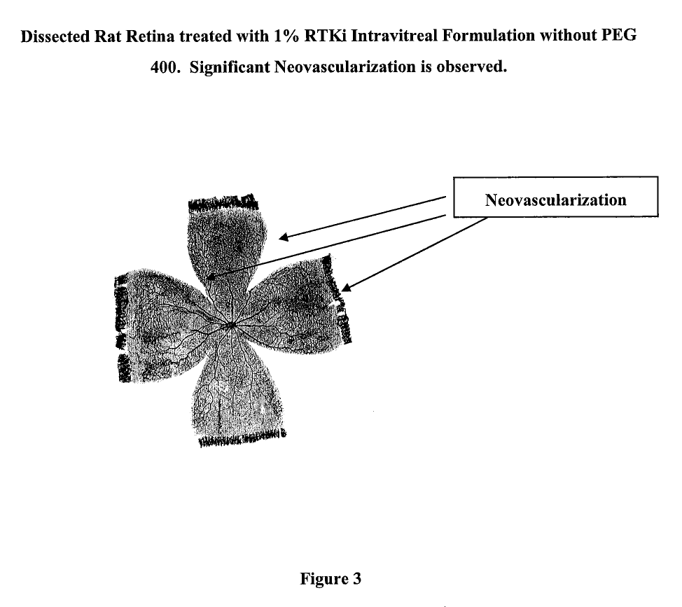

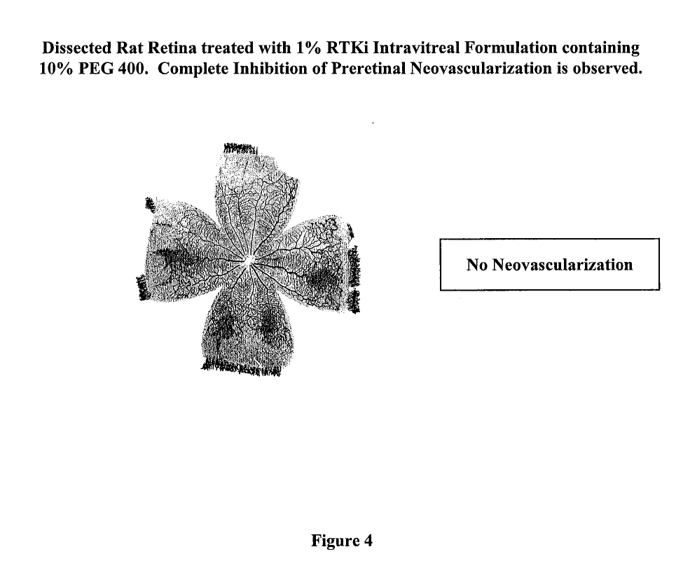

The formulation containing 10% PEG 400 showed complete inhibition upon single intravitreal injection on rat OIR model whereas formulations with 5% PEG 400 and 0% PEG 400 showed 67% and 34% inhibition, respectively (Table 2, FIG. 2). Dissected rat retina clearly illustrates that significant neovascularization occurs in the rat eyes treated with the formulation lacking PEG 400, whereas complete inhibition is observed in the rat eyes treated with formulations containing 10% PEG 400 (FIG. 3 and FIG. 4). Without wishing to be limited by theory or mechanism, it is believed that the efficacy observed is due to higher bioavailability and better distribution of the compound from the formulation containing 10% PEG 400. The compositions of the preferred intravitreal and PJ formulation vehicles and their method of preparation are given in Examples 1 and 2, respectively. The preferred intravitreal formulation for use in the methods of the invention, along with its method of preparation, is provided in Example 3.

TABLE 2
Efficacy of RTKi (N-[4-(3-amino-1H-indazol-4-yl)phenyl]-N′-
(2-fluoro-5-methylphenyl)urea)
Intravitreal Formulations Vs. PEG 400 Concentration in Rat OIR Model

| | % Inhibition | P |
|---|---|---|
| Formulation | (vs vehicle-injected eye) | value |
| 1% RTKi in vehicle with 0% PEG | 34 | 0.198 |
| 400 | | |
| 1% RTKi in vehicle with 5% PEG | 67 | 0.003 |
| 400 | | |
| 1% RTKi in vehicle with 10% PEG | 100 | <0.001 |
| 400 | | |
| 1% RTKi in HPMC/Polysorbate 80 | 63 | 0.010 |
| vehicle | | |

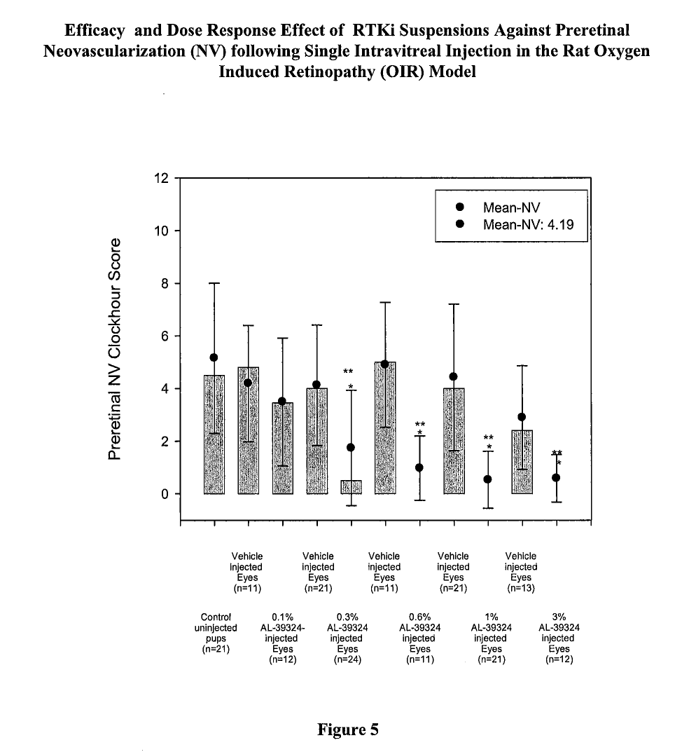

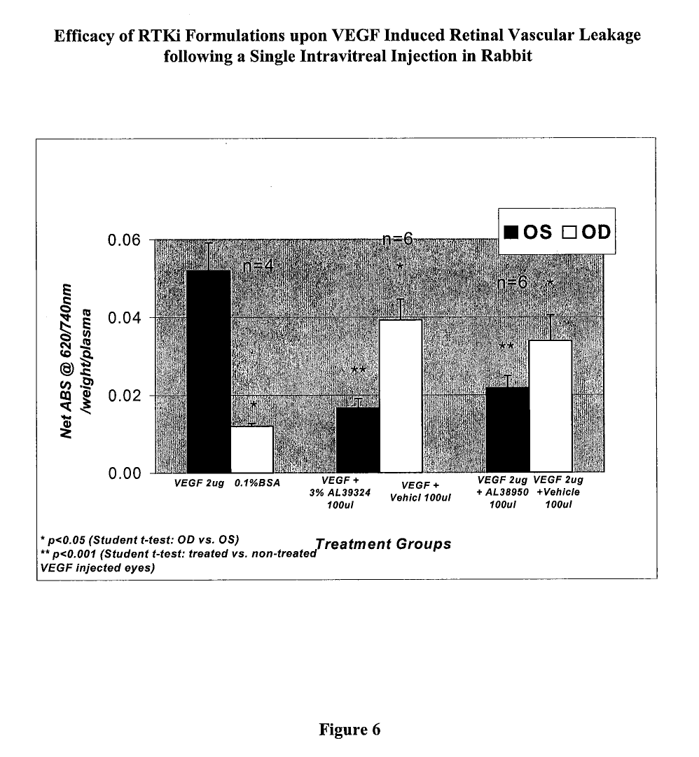

Results of a dose response study in the rat OIR model via intravitreal route using formulations of N-[4-(3-amino-1H-indazol-4-yl)phenyl]-N′-(2-fluoro-5-methylphenyl)urea having concentrations of 3%, 1%, 0.6%, 0.3% and 0.1% are shown in FIG. 5. A formulation of 3% N-[4-(3-amino-1H-indazol-4-yl)phenyl]-N′-(2-fluoro-5-methylphenyl)urea effectively reduced retinal vascular leakage following a single intravitreal injection in rabbit VEGF induced leakage model (FIG. 6).

Pharmaceutical compositions for intravitreal, posterior juxtascleral, periocular and topical administration contain an effective amount of non-ionic surfactant, polysorbate 80, in an amount of from 0.05% to 2%. Preferably, the compositions of the invention will contain from 0.01% to 1% non-ionic surfactant, and most preferably, the composition of the invention will contain 0.5% surfactant.

Although the efficacy of 1% N-[4-(3-amino-1H-indazol-4-yl)phenyl]-N′-(2-fluoro-5-methylphenyl)urea formulation containing 10% PEG 400 is excellent, physical stability of the formulation was not ideal. Therefore, a polymer that acts as a suspending agent is included in the composition to enhance the physical stability of the formulation. A number of polymers, such as hydroxypropyl methyl cellulose (HPMC), hydroxyethyl cellulose (HEC), methyl cellulose, carbopol, polyvinyl alcohol (PVA), polyvinyl pyrrolidone (PVP), xanthan, gum tragacanth, gum acacia, sodium alginate etc. can be used for this purpose.

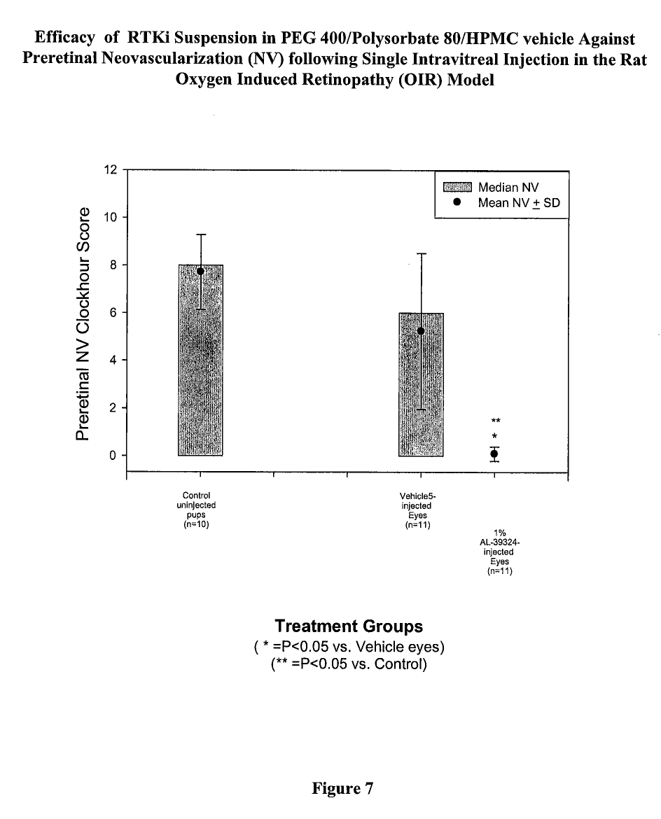

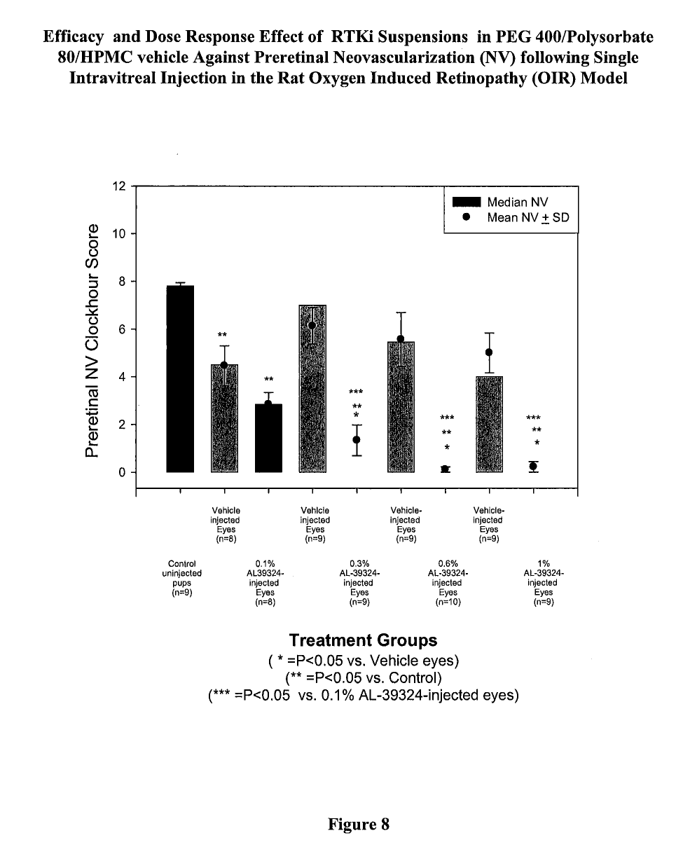

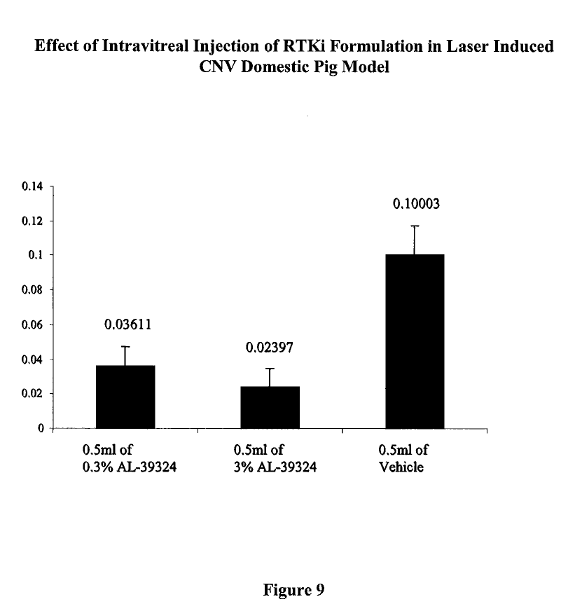

The preferred suspending agent for use in the compositions of the present invention is HPMC 2910 (EM4). Addition of HPMC 2910 substantially increased the physical stability of the formulation, and the formulation shows 100% inhibition upon single intravitreal injection in the rat OIR model (Table 3, FIG. 7). Furthermore, the efficacy results show low standard deviation. The HPMC 2910 (EM4) containing formulations also exhibit excellent dose dependent inhibition (FIG. 8). The formulations also exhibit excellent efficacy in the laser induced mouse CNV model, the rat VEGF model, and the laser induced domestic pig CNV model (FIG. 9).

TABLE 3
Composition of RTKi (N-[4-(3-amino-1H-indazol-4-yl)phenyl]-N′-
(2-fluoro-5-methylphenyl)urea) Intravitreal Formulation

| Ingredient | Amount (w/v, %) |
|---|---|
| RTKi | 0.1-6 |
| PEG 400 | 1-10 |
| Polysorbate 80 | 0.001-0.5 |
| HPMC 2910 | 0.005-1 |
| Dibasic sodium phosphate, dodecahydrate | 0.18-0.5 |
| Sodium hydroxide | q.s. to pH 7.2 |
| Hydrochloric acid | q.s. to pH 7.2 |
| Water for Injection | q.s. to 100% |

The suspending agent will typically be present in the composition of the invention in an amount of from 0.005% to 1%. Preferably, the compositions of the invention will contain from 0.05% to 0.7% suspending agent. Most preferably, the composition of the invention will contain 0.5% suspending agent.

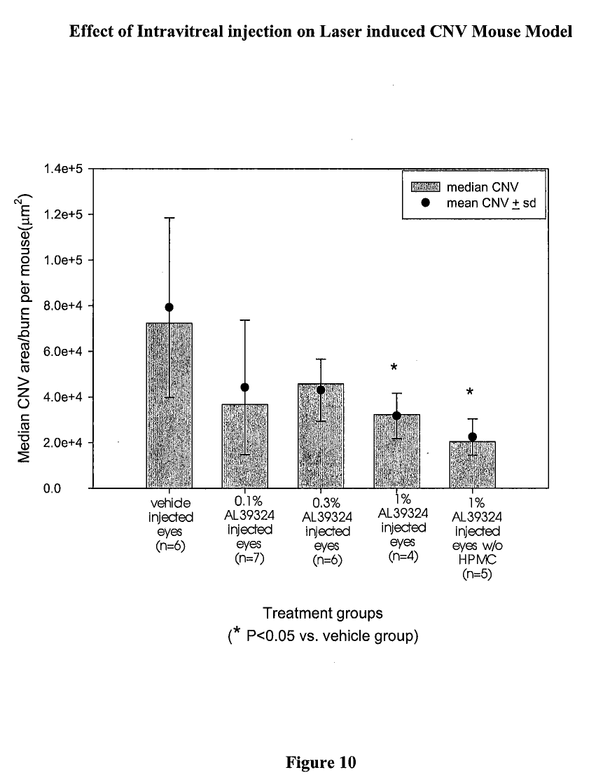

The formulation containing 10% PEG 400 (with or without HPMC) exhibits excellent efficacy upon single intravitreal injection in the laser induced mouse CNV model, shown in FIG. 10.

The formulations for posterior juxtascleral administration, periocular administration or topical administration will preferably be isotonic to avoid irritation and other vascular damage upon administration. Therefore, the formulation for posterior juxtascleral administration contains 5% PEG 400, and tonicity is adjusted by adding a suitable amount of sodium chloride (Table 4). This formulation contains polysorbate 80 as a surfactant. The composition and method of preparation of a preferred formulation for posterior juxtascleral administration is shown in Example 4.

TABLE 4
Composition of RTKi (N-[4-(3-amino-1H-indazol-4-yl)phenyl]-N′-
(2-fluoro-5-methylphenyl)urea) Posterior Juxtascleral,
Periocular and Topical Formulation

| Ingredient | Amount (w/v, %) |
|---|---|
| RTKi | 1-10 |
| PEG 400 | 1-5 |
| Polysorbate 80 | 0.001-0.5 |
| HPMC 2910 | 0.005-1 |
| Dibasic sodium phosphate, dodecahydrate | 0.18-0.5 |
| Sodium Chloride | 0.1-0.5 |
| Sodium hydroxide | q.s. to pH |
| Hydrochloric acid | q.s. to pH |
| Water for Injection | q.s. to 100 |

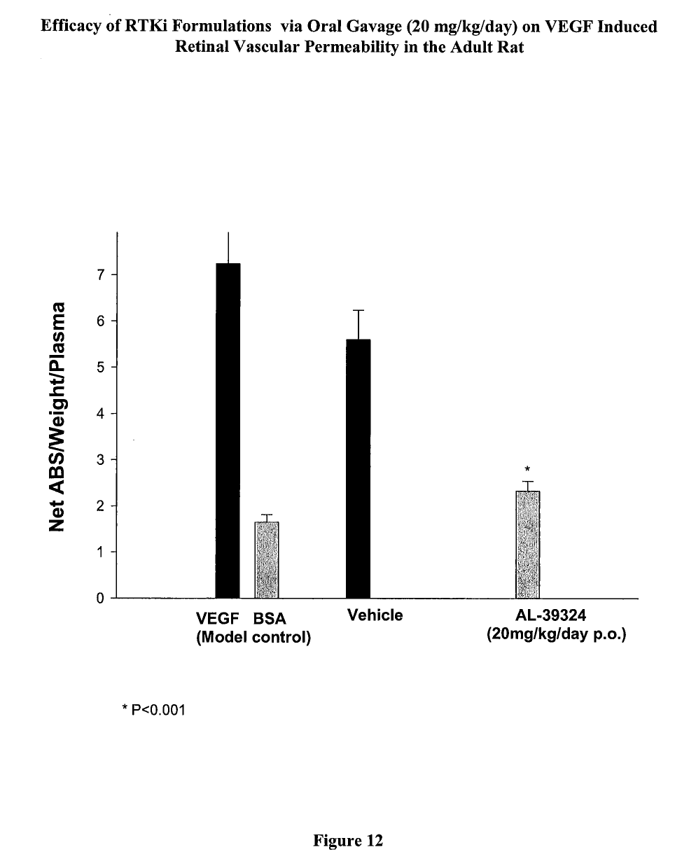

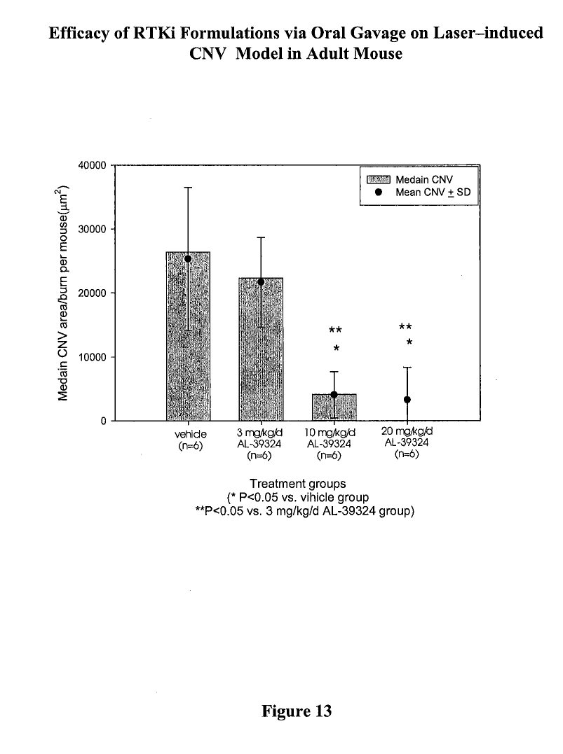

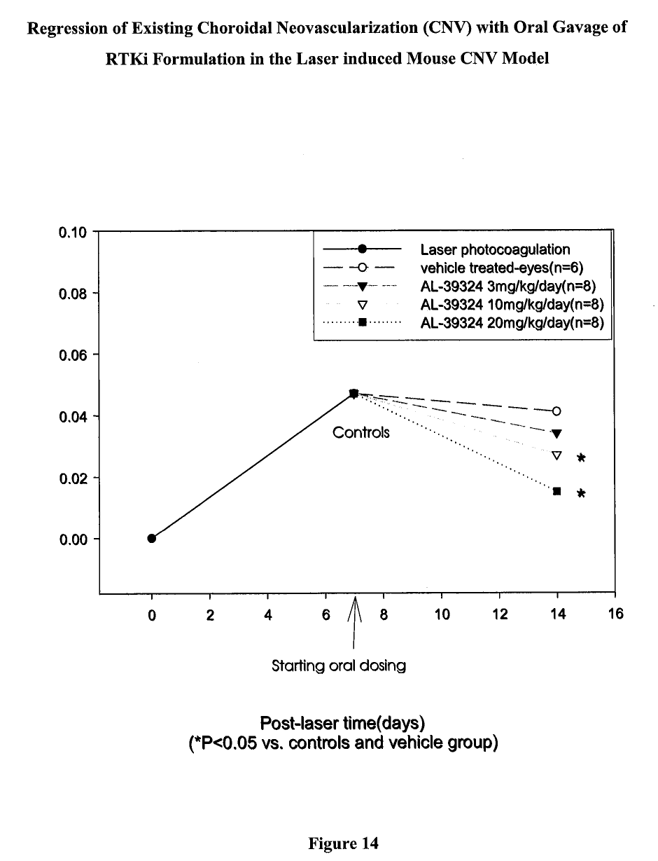

Preferred formulations for oral dosing are prepared in a vehicle containing 1% polysorbate 80, 0.2% HPMC 2910 (EM4), 2% ethanol and purified water (Table 5). The formulation, prepared for oral dosing, shows significant efficacy on blood-retinal barrier breakdown on rat VEGF model (FIG. 12). In the laser induced adult mouse model, systemic delivery of 20 mg/kg/day oral formulation containing N-[4-(3-amino-1H-indazol-4-yl)phenyl]-N′-(2-fluoro-5-methylphenyl)urea provided complete (100%) inhibition of CNV as compared to vehicle treated controls (FIG. 13). Mice treated with 10 mg/kg/day had an 84.3% reduction in CNV, where mice treated with 3 mg/kg/day exhibited no significant inhibition, as compared to vehicle-treated controls. In the same model, mice treated with 20 mg/kg/day and 10 mg/kg/day induced significant regression (↓68.0% and ↓1.8%) of existing CNV, as compared to controls (FIG. 14). The mice treated with 3 mg/kg/day did not cause regression of existing CNV.

The composition and method of preparation of a preferred formulation for oral administration is shown in Example 5.

TABLE EXAMPLE 5
General Composition of RTKi (N-[4-(3-amino-1H-indazol-4-yl)phenyl]-
N′-(2-fluoro-5-methylphenyl)urea) Formulation for Oral Dosing

| | |
|---|---|
| Ingredients | Amount (w/v, %) |

| | |
|---|---|
| RTKi | 0.1-0.5 |
| Polysorbate 80 | 0.001-1 |
| HPMC 2910 | 0.2-0.5 |
| Ethanol | 0.5-2 |
| Sodium hydroxide | q.s. to pH |
| Hydrochloric acid | q.s. to pH |
| Purified water | q.s. to 100 |

The preferred suspending agent for use in the above described posterior juxtascleral and oral formulations is HPMC 2910 (E4M). However, it is contemplated that other polymers, such as hydroxyethyl cellulose (HEC), methyl cellulose, polycarbophil, carbopol, polyvinyl alcohol, polyvinyl pyrrolidone (PVP), xanthan gum, tragacanth gum, acacia etc. can also be used successfully as a suspending agent in the compositions of the invention.

Preferred formulation for topical ocular dosing is isotonic and composition and method of preparation is given in Example 6.

The specific dose level for any particular human or animal depends upon a variety of factors, including the activity of the active compound used, the age, body weight, general health, time and of administration, route of administration and the severity of the pathologic condition undergoing therapy.

The preferred formulations of the invention, for administration via intravitreal injection, periocular administration, posterior juxtascleral administration, topical ocular administration or oral administration, may contain:

An active agent in a therapeutically effective amount;

PEG 200 to PEG 2500 as a co-solvent in an effective amount (from 1 to 30%; 5 to 20% more preferred);

Polysorbate 80 as a surfactant (from 0.1 to 5%; 0.2 to 2% more preferred);

Tonicity agent (particularly for PJ and topical ocular)

Suitable buffer;

Suspending agent (from 0.05% to 1%; 0.05% to 0.5% more preferred).

A preferred formulation for oral dosing of the invention may contain:

An anti-angiogenic agent in a therapeutically effective amount;

Ethyl alcohol co-solvent in an effective amount (from 0.5% to 5%; 1% to 3% more preferred);

Polysorbate 80 as a surfactant (from 0.1% to 5%; 0.5% to 3%);

Suitable buffer;

Suspending agent (from 0.05% to 0.5%; 0.1% to 0.3% more preferred).

Due to the preferred route of administration (IVT or PJ), it is very important that the particle size of the formulations must be small to accomplish good syringibility, as well as comfort. Suspensions with particle size from 1 μm-3 μm are prepared by this compounding procedure. The prepared formulations (for IVT or PJ) exhibit excellent syringibility even when only 2 μL-10 μL of the formulation is injected in the eyes of the animals.

The following examples are included to demonstrate preferred embodiments of the invention. It should be appreciated by those of skill in the art that the techniques disclosed in the examples which represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

This example illustrates the composition and method of preparation of a preferred intravitreal formulation vehicle.

| Ingredient | Amount (w/v, %) |
|---|---|
| PEG 400 | 10 |
| Polysorbate 80 | 0.5 |
| HPMC 2910 | 0.5 |
| Dibasic sodium phosphate, dodecahydrate | 0.18 |
| Sodium hydroxide | q.s. to pH 7.2 |
| Hydrochloric acid | q.s. to pH 7.2 |
| Water for Injection | q.s. to 100 |

In a 250 mL glass container, was added 3.60 g sterile 10% dibasic sodium phosphate, dodecahydrate solution. To it was added 20 g sterile PEG 400 and stirred until a uniform solution formed. To the above solution was added 10 g sterile 10% polysorbate 80 solution and 50 g of sterile 2% stock HPMC 2910 (E4M) solution and stirred well until homogeneous. Sterile water for injection was added to get to 95% of batch size. The solution was stirred at RT for 30 min and pH was adjusted to 7.2. Finally, water for injection was added to get final batch of 200 g.

### EXAMPLE 2

This example describes the composition and method of preparation of PJ, periocular and topical ocular formulation vehicle.

| Ingredient | Amount (w/v, %) |
|---|---|
| PEG 400 | 10 |
| Polysorbate 80 | 0.5 |
| HPMC 2910 | 0.5 |
| Dibasic sodium phosphate, dodecahydrate | 0.18 |
| Sodium Chloride | 0.18 |
| Sodium hydroxide | q.s. to pH 7.2 |
| Hydrochloric acid | q.s. to pH 7.2 |
| Water for Injection | q.s. to 100 |

In a 250 mL glass container, was added 3.60 g sterile 10% dibasic sodium phosphate, dodecahydrate solution. To it was added 10 g sterile PEG 400 and stirred until a uniform solution formed. To the above solution was added 10 g sterile 10% polysorbate 80 solution, 50 g of sterile 2% stock HPMC 2910 (E4M) solution and 7.6 g 5% sodium chloride stock solution and stirred well until homogeneous. Sterile water for injection was added to get to 95% of batch size. The solution was stirred at RT for 30 min and pH was adjusted to 7.2. Finally, water for injection was added to get final batch of 200 g.

### EXAMPLE 3

This example illustrates the composition as well as method of preparation of a representative pharmaceutical formulation for intravitreal ophthalmic administration containing the RTKi, N-[4-(3-amino-1H-indazol-4-yl)phenyl]-N′-(2-fluoro-5-methylphenyl)urea.

| Ingredient | Amount (w/v, %) |
|---|---|
| RTKi | 0.1-10 |
| PEG 400 | 10 |
| Polysorbate 80 | 0.5 |
| HPMC 2910 | 0.5 |
| Dibasic sodium phosphate, dodecahydrate | 0.18 |
| Sodium hydroxide | q.s. to pH |
| Hydrochloric acid | q.s. to pH |
| Water for Injection | q.s. to 100% |

RTKi raw material was sterilized by autoclaving at 121° C. for 45 minutes. Sterile RTKi raw material (1 g) was weighed in a polypropylene container, and to it was added 25 g sterile 2% polysorbate 80 stock solution. The slurry was ball milled at RT for 12 h using Zirconia beads. At the end of ball milling, carefully filtered the suspension through a Buckner funnel and washed the Zirconia beads thoroughly with sterile water. To the above solution, 10 g sterile PEG 400, 3.6 g 5% sterile stock solution of dibasic sodium phosphate, dodecahydrate and 25 g 2% HPMC stock solution were added sequentially. Sufficient amount of sterile water for injection was added to get 95% of batch size. The pH was adjusted to 7.2 and q.s. to 100% of batch size (100 g) by sterile water for injection. The above formulation was intravitreally administered in rat OIR, mouse CNV, rabbit VEGF and pig CNV models, and the results are shown in FIGS. 1, 2, 5, 6, 7, 8 and 10.

### EXAMPLE 4

This example illustrates the composition and preparation of a representative pharmaceutical formulation containing RTKi, N-[4-(3-amino-1H-indazol-4-yl)phenyl]-N′-(2-fluoro-5-methylphenyl)urea, for posterior juxtascleral and periocular administration.

| Ingredients | Amount (w/v, %) |
|---|---|
| RTKi | 5 |
| PEG 400 | 5 |
| Polysorbate 80 | 0.5 |
| HPMC 2910 | 0.5 |
| Dibasic sodium phosphate, dodecahydrate | 0.18 |
| Sodium Chloride | 0.17 |
| Sodium hydroxide | q.s. to pH |
| Hydrochloric acid | q.s. to pH |
| Water for Injection | q.s. to 100% |

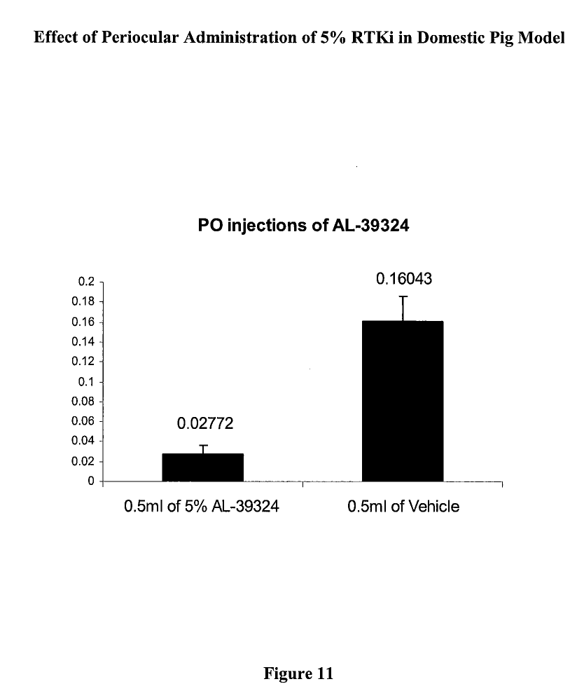

5 g of sterile RTKi raw material was taken in a sterile polypropylene container, and to it was added 50 g of 1% polysorbate 80 stock solution. The slurry was ball milled using Zirconia beads at RT for 12 h. At the end of ball milling, carefully filtered the suspension through a Buckner funnel and wash the Zirconia beads thoroughly with sterile water for injection. To the above solution, 5 g PEG 400, 3.6 g 5% sterile solution of dibasic sodium phosphate, dodecahydrate, 1.7 g of 1% sodium chloride stock solution and 25 g of 2% HPMC stock solution were added sequentially. Sufficient amount of water for injection was added to get 95% of batch size and was stirred at RT for 30 min. Formulation pH was adjusted to 7.2, and q.s. to 100% of batch size (100 g) with sterile water for injection. The above formulation was injected via periocular route in domestic pig CNV model and the result is shown in FIG. 11.

### EXAMPLE 5

This example details the preparation of a representative pharmaceutical composition containing RTKi, N-[4-(3-amino-1H-indazol-4-yl)phenyl]-N′-(2-fluoro-5-methylphenyl)urea, for oral administration.

| Ingredients | Amount (w/v, %) |
|---|---|
| RTKi | 0.5 |
| Polysorbate 80 | 0.5 |
| HPMC 2910 | 0.5 |
| Ethanol | 2 |
| Sodium hydroxide | q.s. to pH |
| Hydrochloric acid | q.s. to pH |
| Purified water | q.s. to 100 |

In a glass container was taken 0.5 g RTKi raw material and to it was added 50 g of 2% polysorbate 80 stock. The slurry was ball milled using Zirconia beads at RT for 12 h. At the end of ball milling, carefully filtered the suspension through a Buckner funnel and washed the Zirconia beads thoroughly with sterile water. To the slurry were added 10 g of 2% HPMC stock solution and 2 g ethanol, and stirred well. Sufficient amount of purified water was then added to get 95% of batch size. The pH was adjusted to 7.2 and q.s. to 100% of batch size with sterile water. This formulation was administrated to adult rats via oral gavage to test VEGF induced vascular permeability and is shown in FIG. 12.

### EXAMPLE 6

This example illustrates the preparation of a representative pharmaceutical formulation containing RTKi, N-[4-(3-amino-1H-indazol-4-yl)phenyl]-N′-(2-fluoro-5-methylphenyl)urea, for topical ocular application.

| Ingredients | Amount (w/v, %) |
|---|---|
| RTKi | 1 |
| PEG 400 | 5 |
| Polysorbate 80 | 0.5 |
| HPMC 2910 | 0.5 |
| Sodium Chloride | 0.25 |
| Sodium hydroxide | q.s. to pH |
| Hydrochloric acid | q.s. to pH |
| Purified water | q.s. to 100 |

1 g sterile RTKi raw material was taken in a sterile polypropylene container, and to it was added 25 g of 2% polysorbate 80 stock solution. The slurry was ball milled using Zirconia beads at RT for 12 h. Carefully filter the suspension through a Buckner funnel at the end of ball milling and wash the Zirconia beads thoroughly with sterile water. To it was added 5 g sterile PEG 400, 3.6 g of 5% sterile solution of dibasic sodium phosphate, dodecahydrate, 5 g of 5% sodium chloride stock solution and 25 g of 2% HPMC stock solution. Sufficient amount of sterile water was added to get 95% of batch size and was stirred at RT for 1 h. The pH was adjusted to 7.2, and q.s. to 100% of batch size (100 g) with sterile water. The above formulation was applied via topical ocular route in rabbit model.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

We claim:

1. An ophthalmic composition for intravitreal injection for treating ocular neovascularization, comprising:
i) an active agent in an amount of from 0.001% to 30%, and
ii) a polyethylene glycol co-solvent having a molecular weight of from 200 to 2500.

2. □□ The ophthalmic composition of claim 1, wherein the active agent is selected from the group consisting of anti-angiogenic agents, anti-inflammatory agents, and anti-vascular permeability agents.

3. The ophthalmic composition of claim 2, wherein the active agent is an anti-angiogenic agent.

4. The ophthalmic composition of claim 3, wherein the anti-angiogenic agent is a multi-targeted receptor tyrosine kinase (RTK) inhibitor.

5. The ophthalmic composition of claim 4, wherein the RTK inhibitor is N-[4-(3-amino-1H-indazol-4-yl)phenyl]-N′-(2-fluoro-5-methylphenyl)urea.

6. The ophthalmic composition of claim 3, wherein the concentration of the anti-angiogenic agent is from 1% to 15%.

7. The ophthalmic composition of claim 1, wherein the co-solvent is PEG 400.

8. The ophthalmic composition of claim 7, wherein the concentration of PEG 400 in the formulation is from 1% to 20%.

9. The ophthalmic composition of claim 1, further comprising polysorbate 80 as a surfactant, wherein the concentration of polysorbate 80 is from 0.001% to 1%.

10. The ophthalmic composition of claim 1, further comprising HPMC, wherein the concentration of the HPMC is from 0.01% to 2%.

11. An ophthalmic composition for posterior juxtasclaral, periocular or topical administration, said composition comprising:
i) an active agent, wherein the concentration of the anti-angiogenic agent is from 0.001% to 30%; and
ii) a polyethylene glycol co-solvent having a molecular weight of from 200 to 2500

12. □ The ophthalmic composition of claim 11, wherein the active agent is selected from the group consisting of anti-angiogenic agents, anti-inflammatory agents, and anti-vascular permeability agents.

13. The ophthalmic composition of claim 12, wherein the active agent is an anti-angiogenic agent.

14. The ophthalmic composition of claim 13, wherein the anti-angiogenic agent is a multi-receptor targeted receptor tyrosine kinase (RTK) inhibitor.

15. The ophthalmic composition of claim 11, wherein the co-solvent is PEG 400 and wherein the concentration of the PEG 400 is from 1% to 20%.

16. The ophthalmic composition of claim 11 further comprising polysorbate 80 as a surfactant, wherein the concentration of the polysorbate 80 is from 0.001% to 1%.

17. The ophthalmic composition of claim 11, further comprising HPMC, wherein the concentration of the HPMC is from 0.01% to 2%.

18. A composition for intravitreal injection for the treatment of ocular neovascularization, said composition comprising:
from 0.001 to 10% of a multi-targeted receptor tyrosine kinase inhibitor;
10% of PEG 400;
0.5% of polysorbate 80; and
0.5% of HPMC 2910.

19. The composition of claim 18, wherein the RTK inhibitor is N-[4-(3-amino-1H-indazol-4-yl)phenyl]-N′-(2-fluoro-5-methylphenyl)urea.
